SEQUENCE LISTING (30 sequences)

SEQ ID NO: 1 - protein, 9 aa, synthetic construct
GSIFDYASM

SEQ ID NO: 2 - protein, 14 aa, synthetic construct
EFVAGINYGSITYY

SEQ ID NO: 3 - protein, 10 aa, synthetic construct
AFFDGDYYLY

SEQ ID NO: 4 - protein, 8 aa, synthetic construct
GNIFYVYM

SEQ ID NO: 5 - protein, 14 aa, synthetic construct
ELVAGIDYGSTTYY

SEQ ID NO: 6 - protein, 14 aa, synthetic construct
AYSPDANYHQAHHV

SEQ ID NO: 7 - protein, 9 aa, synthetic construct
GYIFWYNGM

SEQ ID NO: 8 - protein, 14 aa, synthetic construct
ELVAAIDSGATTYY

SEQ ID NO: 9 - protein, 15 aa, synthetic construct
AVYYYGPSVAYSAHW

SEQ ID NO: 10 - protein, 9 aa, synthetic construct
GTISDYDAM

SEQ ID NO: 11 - protein, 14 aa, synthetic construct
ELVAAIDVGTTTYY

SEQ ID NO: 12 - protein, 11 aa, synthetic construct
ASVPAYGYYYG

SEQ ID NO: 13 - protein, 9 aa, synthetic construct
GNISDYGSM

SEQ ID NO: 14 - protein, 14 aa, synthetic construct
EFVASINQGTSTNY

SEQ ID NO: 15 - protein, 14 aa, synthetic construct
AFVYVTYWSYSWYY

SEQ ID NO: 16 - protein, 9 aa, synthetic construct
GSISYFYVM

SEQ ID NO: 17 - protein, 14 aa, synthetic construct
EFVAGINYGSITYY

SEQ ID NO: 18 - protein, 10 aa, synthetic construct
ATYDYGWLHS

SEQ ID NO: 19 - protein, 9 aa, synthetic construct
GTIFYDDYM

SEQ ID NO: 20 - protein, 14 aa, synthetic construct
ELVAAIAYGAITYY

SEQ ID NO: 21 - protein, 9 aa, synthetic construct
VWYGEEYHV

SEQ ID NO: 22 - protein, 9 aa, synthetic construct
GNISDYYYM

SEQ ID NO: 23 - protein, 14 aa, synthetic construct
EFVAGIDYGSITYY

SEQ ID NO: 24 - protein, 10 aa, synthetic construct
VFPWRITWHV

SEQ ID NO: 25 - protein, 10 aa, synthetic construct
PAKGRIYQGT

SEQ ID NO: 26 - protein, 9 aa, synthetic construct
QNPMTLGNV

SEQ ID NO: 27 - protein, 12 aa, synthetic construct
EELKEPEKNGIS

SEQ ID NO: 28 - protein, 12 aa, synthetic construct
GVRPSNADALVG

SEQ ID NO: 29 - protein, 16 aa, synthetic construct
LGSQDKVMLRPADPRS

SEQ ID NO: 30 - protein, 10 aa, synthetic construct
EAPKVVEEQE

(19) United States
(12) Patent Application Publication
(10) Pub. No.: US 2024/0228598 A1
(43) Pub. Date: Jul. 11, 2024

(54) NANOBODY FOR PREVENTING OR TREATING SCHIZOPHRENIA

(71) Applicant: POSTECH RESEARCH AND BUSINESS DEVELOPMENT FOUNDATION, Pohang-si (KR)

(72) Inventors: Su Jin NOH, Namyangju-si (KR); Jaewoo AHN, Pohang-si (KR)

(21) Appl. No.: 18/403,934

(22) Filed: Jan. 4, 2024

(30) Foreign Application Priority Data
Jan. 11, 2023 (KR) 10-2023-0003813
Aug. 11, 2023 (KR) 10-2023-0105384

(51) Int. Cl. C07K 16/18; A61K 47/68; A61P 25/18

(52) U.S. Cl. CPC C07K 16/18; A61K 47/6803; A61K 47/6843; A61P 25/18; A61K 2039/505; C07K 2317/14; C07K 2317/565; C07K 2317/569

(57) ABSTRACT

The present invention relates to a nanobody or an antigen binding fragment thereof, and a pharmaceutical composition for preventing or treating a mental or neurological disease, which includes the nanobody or antigen binding fragment thereof. The nanobody or antigen binding fragment thereof according to the present invention binds to proteins (i.e., C1, C3, or C4) of the complement system to inhibit or interfere with their activity, thereby preventing a decrease in dendritic spines in nerve cells, and thus effectively improving the positive and negative symptoms of schizophrenia. In particular, the nanobody or antigen binding fragment thereof according to the prevent invention has an excellent advantage in that it may improve the negative symptoms of schizophrenia, which were difficult to treat in the past.

### CROSS-REFERENCE TO RELATED APPLICATION

This application claims priority to and the benefit of Korean Patent Application No. 10-2023-0003813, filed on Jan. 1, 2023 and Korean Patent Application No. 10-2023-0105384, filed on Aug. 11 2023, disclosure of which is incorporated herein by reference in its entirety.

### REFERENCE TO AN ELECTRONIC SEQUENCE LISTING

The contents of the electronic sequence listing (EPL20236479US_Sequence_listing.xml; Size: 27 K bytes; and Date of Creation: Jan. 4, 2024) is herein incorporated by reference in its entirety. The contents of the electronic sequence listing in no way introduces new matter into the specification.

### BACKGROUND

### 1. Field of the Invention

The present invention relates to a nanobody or an antigen binding fragment thereof, and a pharmaceutical composition for preventing or treating a mental or neurological disease, which includes the nanobody or antigen binding fragment thereof.

### 2. Discussion of Related Art

Since the development of chlorpromazine as a dopamine antagonist, which is the first psychotropic drug, it has not deviated from its treatment target as a dopamine D2 receptor antagonist for nearly 70 years. However, this drug has extrapyramidal side effects. Afterward, second-generation psychotropic drugs were developed, which have limitations in that they have side effects that increase the risk of metabolic syndrome such as diabetes, etc. Also, there is no currently approved therapy to treat the negative symptoms of schizophrenia.

To date, several hypotheses have been proposed regarding the etiology of schizophrenia, including the dopamine hypothesis, NMDA receptor hypofunction hypothesis, opinions based on disrupted-in-schizophrenia 1 (DISC1), and the like. However, in 2014, the American Schizophrenia Group published papers on a large-scale genomic analysis of schizophrenia patients, which had a significant impact on the schizophrenia academic community. Most of the hypotheses previously believed to be true have collapsed, and it was known according to the above-published papers that a protein called complement 4 (C4), which is associated with immunity, increases the most. It is known that C4 is a type of major histocompatibility complex (MHC), and when expressed in dendritic spines of neurons, microglia recognize it to engage in phagocytosis. This is called synaptic pruning, and this phenomenon occurs during adolescence during neurodevelopment. Therefore, this may explain the onset of schizophrenia after adolescence, and this possibility has been proven by a decrease in dendritic spines in animal models overexpressing C4. Furthermore, it has suggested that it is associated with the negative symptoms of schizophrenia.

Therefore, in consideration of the situation in which schizophrenia therapeutics are needed to develop in a completely different way than before, the present inventors found that nanobodies with modified antibodies may be used to treat schizophrenia because they inhibit and interfere with the activity of proteins of the complement system to prevent a decrease in dendritic spines in neurons. Therefore, the present invention was completed based on the above finding.

### SUMMARY OF THE INVENTION

The present invention is directed to providing a nanobody or an antigen binding fragment thereof.

The present invention is also directed to providing a polynucleotide encoding the nanobody.

The present invention is also directed to providing an expression vector including the polynucleotide.

The present invention is also directed to providing a transformant having the expression vector introduced thereto.

The present invention is also directed to providing a nanobody-drug conjugate in which a drug is bound to the nanobody or antigen binding fragment thereof.

The present invention is also directed to providing a pharmaceutical composition for preventing or treating a mental or neurological disease, which includes the nanobody or antigen binding fragment thereof.

According to an aspect of the present invention, there is provided a nanobody or an antigen binding fragment thereof.

According to another aspect of the present invention, there is provided a polynucleotide encoding the nanobody or antigen binding fragment thereof.

According to still another aspect of the present invention, there is provided an expression vector including the polynucleotide.

According to yet another aspect of the present invention, there is provided a transformant having the expression vector introduced thereto.

According to yet another aspect of the present invention, there is provided a nanobody-drug conjugate in which a drug is bound to the nanobody or antigen binding fragment thereof.

According to yet another aspect of the present invention, there is provided a pharmaceutical composition for preventing or treating a mental or neurological disease, which includes the nanobody or antigen binding fragment thereof.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows the results of confirming the target selectivity of nanobodies;

FIG. 2 shows the results of confirming the target selectivity of the nanobodies;

FIG. 3 shows the results of confirming that the nanobody of the present invention penetrating through the blood-brain barrier (BBB);

FIG. 4 shows the results of confirming cellular model of schizophrenia of the present invention;

FIG. 5 shows the results of confirming the effect of the nanobody on the dendritic spines of the present invention in a cellular model of schizophrenia;

FIG. 6 shows the results of confirming that the nanobody of the present invention penetrating through the blood-brain barrier in an animal model (in vivo);

FIG. 7 shows the results of confirming the effect of the nanobody of the present invention on negative symptoms in an animal model of schizophrenia (social preference);

FIG. 8 shows the results of confirming the effect of the nanobody of the present invention on negative symptoms in the animal model of schizophrenia (social recognition); and

FIG. 9 shows the results of confirming the effect of the nanobody of the present invention on positive symptoms in the animal model of schizophrenia.

### DETAILED DESCRIPTION OF EXEMPLARY EMBODIMENTS

Hereinafter, the present invention will be described in further detail.

The present invention provides a nanobody or an antigen binding fragment thereof, which includes: (a) heavy-chain CDR1 including a sequence of SEQ ID NO: 1, heavy-chain CDR2 including a sequence of SEQ ID NO: 2, and heavy-chain CDR3 including a sequence of SEQ ID NO: 3; and light-chain CDR1 including a sequence of SEQ ID NO: 4, light-chain CDR2 including a sequence of SEQ ID NO: 5, and light-chain CDR3 including a sequence of SEQ ID NO: 6, (b) heavy-chain CDR1 including a sequence of SEQ ID NO: 7, heavy-chain CDR2 including a sequence of SEQ ID NO: 8, and heavy-chain CDR3 including a sequence of SEQ ID NO: 9; and light-chain CDR1 including a sequence of SEQ ID NO: 10, light-chain CDR2 including a sequence of SEQ ID NO: 11, and light-chain CDR3 including a sequence of SEQ ID NO: 12, (c) heavy-chain CDR1 including a sequence of SEQ ID NO: 13, heavy-chain CDR2 including a sequence of SEQ ID NO: 14, and heavy-chain CDR3 including a sequence of SEQ ID NO: 15; and light-chain CDR1 including a sequence of SEQ ID NO: 16, light-chain CDR2 including a sequence of SEQ ID NO: 17, and light-chain CDR3 including a sequence of SEQ ID NO: 18, or (d) heavy-chain CDR1 including a sequence of SEQ ID NO: 19, heavy-chain CDR2 including a sequence of SEQ ID NO: 20, and heavy-chain CDR3 including a sequence of SEQ ID NO: 21; and light-chain CDR1 including a sequence of SEQ ID NO: 22, light-chain CDR2 including a sequence of SEQ ID NO: 23, and light-chain CDR3 including a sequence of SEQ ID NO: 24.

In the present invention, C4 is a protein present in the complement system that is degraded from complement 1 (C1) or is overexpressed on its own for various reasons. C1 is also secreted from astrocytes due to inflammatory responses, and C1q, which is a subunit of secreted C1, attaches to the dendritic spines of nerve cells. The C1q attaches only to damaged cell membranes, which makes it possible to distinguish whether or not microglia will engage in phagocytosis. Also, C4 is decomposed into C3. In this case, microglia have a receptor that recognizes C3, and through this, phagocytosis occurs. Therefore, if C4, C1, C3, or the enzymes that decompose them can be inhibited or blocked, the progression of schizophrenia may be delayed or treated, but such a treatment method has not yet been attempted in animal models of schizophrenia or patients with schizophrenia.

Meanwhile, among biopharmaceuticals, antibody therapeutic agents with high target selectivity have been widely used in recent years, but their introduction in the field of brain diseases has been significantly delayed. This is because antibodies cannot pass through the blood-brain barrier. This is the fundamental reason why the antibodies cannot pass through the blood-brain barrier (BBB) is due to receptors that recognize the Fc regions of the antibodies in the BBB. Because antibodies may specifically block the action of target proteins at lower concentrations than general compounds, biopharmaceuticals such as antibodies have been used for many diseases, including cancer.

Therefore, in the present invention, the present inventors attempted to use nanobodies modified from antibodies to effectively inhibit C1q, C4, C3, and the like. Meanwhile, in the field of brain diseases, attempts to use antibodies are being delayed because it is difficult for the antibodies to pass through the blood-brain barrier. Therefore, in the present invention, a therapeutic agent for schizophrenia was developed in the form of a small-molecule nanobody in which the Fc region was removed, which is one of the reasons why it is difficult for antibodies to pass through the blood-brain barrier.

In the present invention, the nanobody is a protein molecule that includes an immunoglobulin molecule that is immunologically reactive with a specific antigen, and acts as a receptor that specifically recognizes the antigen. Thus, the nanobody refers to a structure excluding the Fc region from an antibody, which is a conventional immunoglobulin molecule.

In the present invention, an immunoglobulin has a heavy chain and a light chain, and each of the heavy and light chains includes a constant region and a variable region. The variable regions of the light and heavy chains include three variable regions called complementarity-determining regions (hereinafter referred to as “CDRs”) and four framework regions (hereinafter referred to as “FRs”). The CDRs of each chain mainly function to bind to the epitope of an antigen, and are typically named CDR1, CDR2, and CDR3 sequentially starting from the N-terminus. Also, the FRs of each chain may be named FR1, FR2, FR3, and FR4 sequentially starting from the N-terminus.

In the present invention, the variable regions of the heavy and light chain may be named ‘VH’ and ‘VL,’ respectively, and the CDRs of the heavy chain may be named ‘VH-CDR1,’ ‘VH-CDR2,’ and ‘VH-CDR3,’ respectively, and the CDRs of the light chain may be named ‘VL-CDR1,’ ‘VL-CDR2,’ and ‘VL-CDR3,’ respectively. Also, the FRs of the heavy chain may be named ‘VH-FR1,’ ‘VH-FR2,’ ‘VH-FR3,’ and ‘VH-FR4,’ respectively, and the FRs of the light chain may be named ‘VL-FR1,’ ‘VL-FR2,’ ‘VL-FR3,’ and ‘VL-FR4,’ respectively.

In the present invention, the antigen binding fragment refers to a fragment that possesses an antigen binding function, and includes Fd, Fab, Fab′, Fv, and F(ab′)2 but excludes Fc. The Fc refers to the tail portion of an antibody that interacts with a cell surface receptor called an Fc receptor. The Fd refers to the heavy-chain portion included in the Fab fragment, and F(ab′)2 refers to a fragment including Fd, Fab, Fab′, and Fv. The Fab has a structure that includes the variable regions of the light and heavy chains, the constant region of the light chain, and the first constant region (a CH1 domain) of the heavy chain, and has one antigen binding site. Fab′ differs from Fab in that it has a hinge region containing one or more cysteine residues at the C terminus of the CHI domain of the heavy chain. The F(ab′)2 antibody is produced when the cysteine residues in the hinge region of Fab′ form a disulfide bond. Fv (a variable fragment) refers to the minimum antibody fragment containing only the variable regions of the heavy and light chains. A disulfide-stabilized Fv antibody fragment (dsFv) has the variable regions of the heavy and light chains connected via a disulfide bond, and single-chain Fv (scFv) generally has the variable regions of the heavy and light chains covalently connected through a peptide linker. Such antibody fragments may be obtained using proteolytic enzymes (for example, an Fab fragment may be obtained by restriction digestion of the entire antibody with papain, and an F(ab′)2 fragment may be obtained by digestion of the entire antibody with pepsin). Preferably, the antibody fragment may be constructed through genetic recombination technology.

In the present invention, the nanobody may be an antibody in which the Fc region is removed. When the Fc region is removed, the nanobody solves the problem of not penetrating through the blood-brain barrier (BBB) due to receptors that recognize the Fc regions of conventional antibodies. As a result, the nanobody may penetrate through the blood-brain barrier.

In the present invention, the nanobody may prevent a decrease in dendritic spines in neurons.

In the present invention, the variable and constant regions of the nanobody may have the same or different origins, and the variable and constant regions excluding the CDRs may have the same or different origins.

In the present invention, the nanobody may bind to any one or more selected from the group consisting of complement 1 (C1), complement 3 (C3), and complement 4 (C4).

In the present invention, the binding to one or more selected from the group consisting of C1, C3, and C4 means that activity may be inhibited by binding to C1, C3, or C4.

In the present invention, the nanobody may be easily prepared using known antibody preparation techniques. For example, the antibody preparation may be performed by using yeast surface display, and the like. The yeast surface display is a technique for expressing proteins on the surface of yeast cells. Because yeast uses a eukaryotic protein expression system, the use of this technique causes post-translational modification of the expressed proteins, which makes it possible to obtain recombinant proteins that are closer to human proteins compared to phage or ribosome display methods. Also, the yeast display technique has an advantage in that clones are selected through fluorescence-activated cell sorting (FACS), thereby enabling quantitative analysis simultaneously with a selection process.

In the present invention, the nanobody or antigen binding fragment thereof includes one or more CDR sequences having a sequence identity of at least 80% (e.g., at least 85%, 88%, 90%, 91%, 92%, 93%, 94%, 95%, 96%, 97%, 98%, 99%) to those listed in Table 1. Accordingly, those skilled in the art to which the present invention pertains may modify the CDR sequences provided in Table 1 so that the CDR sequences include one or more amino acid substitutions in order to provide improved biological activity, such as binding to any one or more selected from the group consisting of C1, C3 and C4, and the modified amino acid sequences also fall within the scope of the present invention.

Also, the present invention provides a polynucleotide encoding the nanobody or antigen binding fragment thereof.

In the present invention, the polynucleotide refers to a DNA molecule, an RNA molecule, or an analog thereof. The polynucleotide includes, but is not limited to, DNA molecules, such as cDNA, genomic DNA, or synthetic DNA, and RNA molecules, such as guide RNA, messenger RNA, or synthetic RNA. Moreover, the polynucleotide includes single-stranded and double-stranded forms.

Also, the present invention provides an expression vector containing the polynucleotide.

In the present invention, the expression vector may be a vector capable of replicating and/or expressing the polynucleotide in eukaryotic or prokaryotic cells, which include, but are not particularly limited to, mammalian cells (e.g., human, monkey, rabbit, rat, hamster, mouse cells, and the like), plant cells, yeast cells, insect cells, or bacterial cells (e.g., Escherichia coli (E. coli)). Preferably, the expression vector may be a vector that is operably linked to an appropriate promoter so that the nucleotide can be expressed in a host cell, and includes at least one selection marker. As one example, the polynucleotide may be introduced into a phage, plasmid, cosmid, mini-chromosome vector, viral vector, or retroviral vector.

In the present invention, the expression vector may be an expression vector containing a polynucleotide encoding the heavy or light chain of the nanobody, or an expression vector containing both polynucleotides encoding the heavy and light chain.

In addition, the present invention provides a transformant into which the vector is introduced.

In the present invention, the transformant is not particularly limited thereto, but may be transformed E. coli as a host cell into which the expression vector has been introduced. Preferably, the E. coli may be BL21(DE3).

In the present invention, the introduction refers to a method of delivering a vector containing a polynucleotide encoding the nanobody to a host cell. Such introduction may be performed using several methods known in the art, including a calcium phosphate-DNA coprecipitation method, a DEAE-dextran-mediated transfection method, a polybrene-mediated transfection method, an electric shock method, a microinjection method, a liposome fusion method, a Lipofectamine method, a protoplast fusion method, and the like.

Also, the present invention provides a nanobody-drug conjugate in which a drug is bound to the nanobody or antigen binding fragment thereof.

In the present invention, the nanobody-drug conjugate refers to a substance in which a drug is bound to a nanobody by taking advantage of the target specificity, non-toxicity during blood circulation, and pharmacokinetic advantages of the nanobody.

In the present invention, the drug refers to a substance that may be directly or indirectly bound to a nanobody to effectively treat diseases targeted by the nanobody. The nanobody-drug conjugate may be prepared using various nanobody-drug conjugate preparation methods known in the art.

Further, the present invention provides a pharmaceutical composition for preventing or treating a mental or neurological disease, which includes the nanobody or antigen binding fragment thereof.

In the present invention, the prevention may mean any action of suppressing or delaying the onset of mental or neurological diseases by administering the composition.

In the present invention, the pharmaceutically acceptable carrier refers to a carrier or diluent that does not inhibit the biological activity and properties of the administered compound without irritating the living organisms. In compositions formulated as liquid solutions, the pharmaceutically acceptable carrier is sterile and biocompatible, and may include saline, sterile water, Ringer's solution, buffered saline, an albumin injection solution, a dextrose solution, a maltodextrin solution, glycerol, ethanol, and a combination of one or more thereof. When necessary, other conventional additives such as antioxidants, buffers, bacteriostatic agents, and the like may be added. Also, diluents, dispersants, surfactants, binders, and lubricants may be additionally added to formulate injectable formulations (such as aqueous solutions, suspensions, emulsions, and the like), pills, capsules, granules, or tablets.

In the present invention, the pharmaceutical composition may be in various oral or parenteral dosage forms. When formulated, it is prepared using commonly used diluents or excipients such as fillers, extenders, binders, wetting agents, disintegrants, surfactants, and the like. Solid preparations for oral administration include tablets, pills, powders, granules, capsules, and the like. These solid preparations may be prepared by mixing one or more compounds with at least one excipient such as starch, calcium carbonate, sucrose, or lactose, gelatin, and the like. In addition to simple excipients, lubricants such as magnesium stearate, talc, and the like are also used. Liquid preparations for oral administration include suspensions, oral liquids, emulsions, syrups, and the like. In addition to commonly used simple diluents such as water and liquid paraffin, the liquid preparations may include various excipients such as wetting agents, sweeteners, fragrances, preservatives, and the like. Preparations for parenteral administration include sterile aqueous solutions, non-aqueous solvents, suspensions, emulsions, freeze-dried preparations, and suppositories. Propylene glycol, polyethylene glycol, vegetable oils such as olive oil, injectable esters such as ethyl oleate, and the like may be used as the non-aqueous solvents and suspensions. Witepsol, Macrogol, Tween 61, cacao butter, laurin butter, glycerogelatin, and the like may be used as a base for suppositories.

In the present invention, the pharmaceutical composition may have any one formulation selected from the group consisting of tablets, pills, powders, granules, capsules, suspensions, oral liquids, emulsions, syrups, sterile aqueous solutions, non-aqueous solvents, suspensions, emulsions, freeze-dried preparations, and suppositories.

In the present invention, the composition may be administered in a pharmaceutically effective amount. The pharmaceutically effective amount refers to an amount sufficient to treat a disease with a reasonable benefit/risk ratio applicable to medical treatment, and the effective dose level may be determined according to factors including the type of subject, the severity of the disease, the age and sex of a patient, the activity of a drug, the sensitivity to the drug, the time of administration, the route of administration, and the excretion rate, the duration of treatment, concurrently used drugs , and other factors well known in the medical field. The composition of the present invention may be administered as an individual therapeutic agent or in combination with other therapeutic agents, and may be administered sequentially or simultaneously with conventional therapeutic agents. Also, the composition of the present invention may be administered in single or multiple doses. Considering all of the above factors, it is important to administer an amount that can achieve the maximum effect with the minimum amount without any side effects, and the amount may be easily determined by those skilled in the art.

In the present invention, the mental disease means a pathological mental condition that affects a person's thoughts, emotions, and behavior, and refers to a general term for conditions in which mental functions are impaired. The mental disease includes schizophrenia, depression, anxiety disorders, post-traumatic stress disorder (PTSD), panic disorders, autism spectrum disorders, attention deficit hyperactivity disorder (ADHD), and the like, but the present invention is not limited thereto.

Also, the neurological disease includes Alzheimer's disease, mild cognitive impairment, dementia, and the like, but the present invention is not limited thereto.

Meanwhile, because the corresponding features may be substituted in the above-mentioned parts, the description thereof is omitted.

Hereinafter, examples of the present invention will be described in detail to help the understanding of the present invention. However, it should be understood that the following examples can be modified in many different ways, and the scope of the present invention should not be construed as limited to the following examples. Thus, the examples of the present invention are provided to more fully describe the present invention to those skilled in the art.

### Example 1: Preparation of Nanobody of the Present Invention

The nanobodies of the present invention were prepared using a yeast surface display method using the peptides shown in Table 1 below. Here, the selected DNA was cloned into a GST vector (pGEX4) and synthesized. Thereafter, proteins were extracted using a GST tag.

First, the nanobodies of the present invention were selected against the peptides using a yeast surface display method using the peptides in Table 2 below as an antigen in order to target a surface-exposed loop portion in the structure of the complement protein. Here, when the nanobodies were selected, the nanobodies were selected only by yeast surface display without antigen injection into animals (camels) based on a library created based on the CDR sequences of nanobodies registered on the PDB server. Specifically, a process of preparing the nanobody of the present invention is as follows.

After a frozen library yeast stock was thawed, it was inoculated into a Trp +glucose medium, and underwent recovery and preculturing (30° C./220 rpm/overnight). After the OD of precultured cells was measured, 5×10⁹ yeast cells, which are 10 times the number of libraries, were inoculated into a Trp +glucose medium, and cultured for 4 to 6 hours. Next, the medium was replaced with a fresh Trp +glucose medium, and cultured (25° C./220 rpm/1 to 2 days). The yeast cells were harvested in an amount that was 10 times the number of libraries in which the nanobodies were expressed on the surface, resuspended in a selection buffer, mixed with the antigen peptides in Table 2, and then incubated at 4° C. for an hour. Magnetic associated cell separation (MACS) was performed using streptavidin microbeads that bind to biotin at the N-terminus of the antigen peptide in order to select yeast strains having nanobodies interacting with the antigen peptide. Then, the above-described process was performed two more times to exclude yeast having weakly bound nanobodies. After the MACS process was performed three times, the yeast strains were labeled with antibodies against the HA tag at the C-termini of the nanobodies and the biotin of the antigen peptides (each stained with a different fluorescent material). Thereafter, only the yeast strains showing high fluorescence for both the HA tag and the antigen peptides were isolated using flow cytometry (FACS), plated on a YPD plate, and then cultured for 2 to 3 days at 30° C. Then, the binding in each colony was checked using flow cytometry (FACS). CDR sequences of the nanobodies capable of binding to the antigen peptides were obtained through colony PCR and their sequencing (Meanwhile, a Trp +glucose/+galactose medium containing 3.8 g of −Trp drop-out media supplements (US Biological), 6.7 g of yeast nitrogen base, 10 mL of Pen-Strep (10,000 units/mL stock), 20 g of glucose/galactose (glucose for normal growth and galactose for induction of nanobodies), pH 6.0 was used, and a selection buffer containing 20 mM HEPES, pH 7.5, 150 mM sodium chloride, 0.1% (w/v) bovine serum albumin, and 5 mM maltose was used).

The gene (DNA) sequences of the nanobodies selected by the yeast surface display through the above-described process are shown in Table 1 below. The selected gene DNA was cloned into a vector for E. coli expression and N-terminal GST fusion, expressed in E. coli, and purified to produce nanobody proteins.

TABLE 1

| Target | Nb No. | CDR1 | CDR2 | CDR3 |
|---|---|---|---|---|
| Clq #1 | Nb001 | GSIFDYASM | EFVAGINYGSITYY | AFFDGDYYLY |
| | | (SEQ ID NO: 1) | (SEQ ID NO: 2) | (SEQ ID NO: 3) |
| | Nb076 | GNIFYVYM | ELVAGIDYGSTTYY | AYSPDANYHQAHHV |
| | | (SEQ ID NO: 4) | (SEQ ID NO: 5) | (SEQ ID NO: 6) |
| Clq #2 | Nb020 | GYIFWYNGM | ELVAAIDSGATTYY | AVYYYGPSVAYSAHW |
| | | (SEQ ID NO: 7) | (SEQ ID NO: 8) | (SEQ ID NO: 9) |
| | Nb024 | GTISDYDAM | ELVAAIDVGTTTYY | ASVPAYGYYYG |
| | | (SEQ ID NO: 10) | (SEQ ID NO: 11) | (SEQ ID NO: 12) |
| C3 #1 | Nb084 | GNISDYGSM | EFVASINQGTSTNY | AFVYVTYWSYSWYY |
| | | (SEQ ID NO: 13) | (SEQ ID NO: 14) | (SEQ ID NO: 15) |
| C3 #2 | Nb037 | GSISYFYVM | EFVAGINYGSITYY | ATYDYGWLHS |
| | | (SEQ ID NO: 16) | (SEQ ID NO: 17) | (SEQ ID NO: 18) |
| C4 | Nb094 | GTIFYDDYM | ELVAAIAYGAITYY | VWYGEEYHV |
| | | (SEQ ID NO: 19) | (SEQ ID NO: 20) | (SEQ ID NO: 21) |
| C4b | Nb066 | GNISDYYYM | EFVAGIDYGSITYY | VFPWRITWHV |
| | | (SEQ ID NO: 22) | (SEQ ID NO: 23) | (SEQ ID NO: 24) |

TABLE 2

| No. | Amino acid sequence |
|---|---|
| Clq #1 | PAKGRIYQGT (SEQ ID NO: 25) |
| Clq #2 | QNPMTLGNV (SEQ ID NO: 26) |
| C3 #1 | EELKEPEKNGIS (SEQ ID NO: 27) |
| C3 #2 | GVRPSNADALVG (SEQ ID NO: 28) |
| C4 #1 | LGSQDKVMLRPADPRS (SEQ ID NO: 29) |
| C4 #2 | EAPKVVEEQE (SEQ ID NO: 30) |

### Example 2: Confirmation of Target Selectivity of Nanobody of the Present Invention

A surface plasmon resonance (SPR) assay was performed to confirm target selectivity for each nanobody. When a target protein (C1q. C3, or the like) is attached to the surface of gold and a nanobody is allowed to flow along the surface of gold, the polarization angle changes when the nanobody is attached to the target protein. This allows the degree of interaction between the two proteins to be determined.

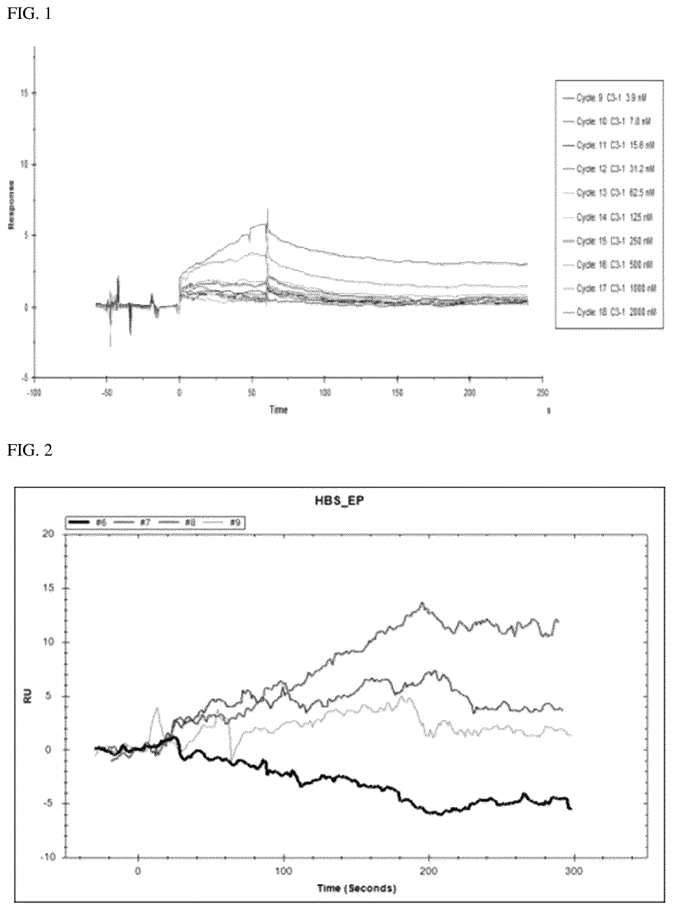

As a result, it can be seen the C3-1 nanobody reacts to the C3 protein in a concentration-dependent manner. The C3-2 nanobody also showed a concentration-dependent reaction, but the degree of reaction was weak. Also, it can be seen that when the C1q nanobodies (#6: Nb001, #7: Nb020, #8: Nb076, #9: Nb024) were allowed to flow along the gold foil attached to the C1q protein, #8 reacted most strongly. Therefore, based on the above results, the present inventors attempted to select the nanobodies with relatively strong target selectivity among several nanobodies according to the present invention and use them in this experiment (FIGS. 1 and 2).

### Example 3: Confirmation of Nanobody of the Present Invention Penetrating Through Blood-brain Barrier (BBB)

A blood-brain barrier model was created to confirm whether the poor blood-brain barrier (BBB) penetration of the nanobodies, which is a drawback of conventional antibody therapeutics, was improved. While typical blood vessels are composed of vascular endothelial cells and pericytes, the blood-brain barrier further includes astrocytes, making it difficult for substances entering the brain to pass therethrough. Therefore, to create an environment similar to the blood-brain barrier, a Transwell insert having a pore size of 3.0 μm was placed in a 12-well plate, and vascular endothelial cells were attached at the upper side, the environment of which was similar to the inside of a blood vessel, and pericytes and astrocytes were attached at the lower side, the environment of which was similar to the outside of the blood vessel and brain parenchymal cells. To check whether this model was well created, the transepithelial electrical resistance (TEER) value was measured.

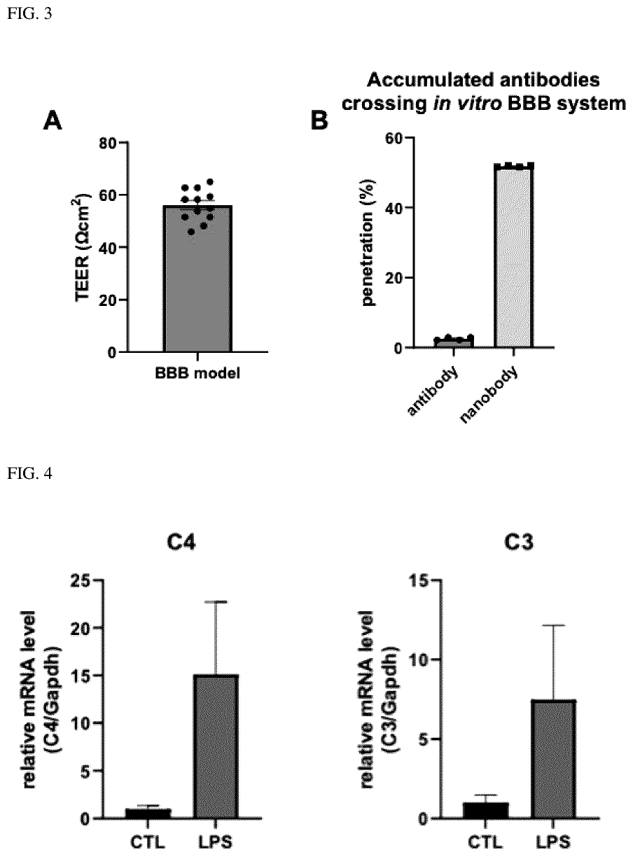

As a result, values similar to those in various documents regarding the blood-brain barrier were obtained, and a transmittance of approximately 50% was observed when the nanobody of the present invention was placed above the Transwell insert in this blood-brain barrier model (FIG. 3).

### Example 4: Confirmation of Effect of Nanobody of the Present Invention in Cell Model of Schizophrenia

To confirm the therapeutic effect of the nanobody of the present invention on schizophrenia, a cell model of schizophrenia was created. One of the characteristic etiologies of schizophrenia is a decrease in dendritic spines. For this purpose, primary neuronal cells were cultured. Because such a decrease in dendritic spines is induced from microglia, neurons and microglia were cultured together, and then treated with LPS to increase a schizophrenia gene (i.e., complement 4).

As a result, as shown in FIGS. 4 and 5, it was confirmed that the mRNA levels of the complements C4 and C3 increased when the cells were treated with 1 μg/mL of LPS. Accordingly, when the nanobody of the present invention was pretreated, it was confirmed that the C1q, C4, and C3 nanobodies (antibodies) all prevented the decrease in dendritic spines (FIGS. 4 and 5).

### Example 5: Confirmation of Nanobody of the Present Invention Passing Through Blood-brain Barrier in Animal Model (in vivo)

To confirm whether the nanobody of the present invention passes through the blood-brain barrier (BBB) in an actual animal, 10 μg/kg of the nanobody was intraperitoneally administered to mice, and the brain was extracted 2 hours later, and analyzed through immunohistochemistry. To confirm whether the nanobody passes through the blood-brain barrier to enter parenchymal cells, it was stained with a NeuN (nerve cell) antibody and confirmed through the GST tag attached to the nanobody.

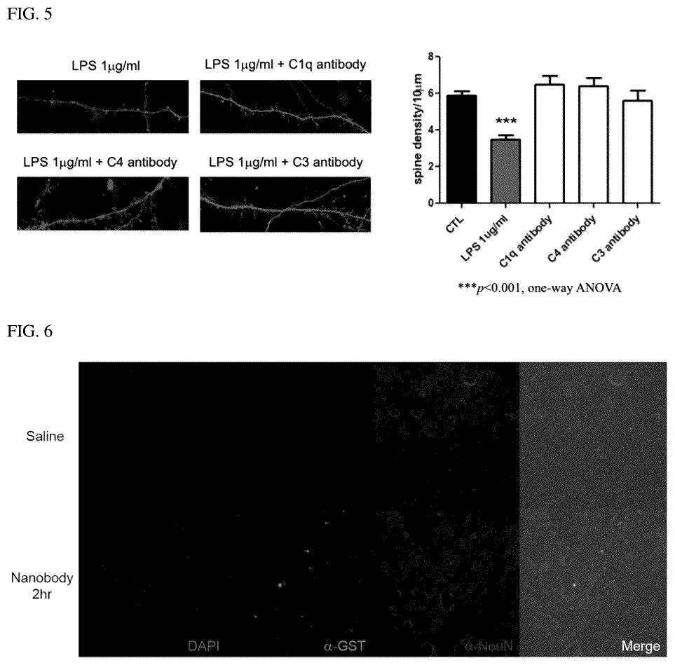

As a result, it can be seen that the GST-nanobody displaying green fluorescence was not present in the blood vessels but present near nerve cells (FIG. 6).

### Example 6: Confirmation of Effect of Nanobody of the Present Invention on Negative Symptoms in Animal Model of Schizophrenia (in vivo), Social Preferncee (1)

To induce an animal model of schizophrenia, 3 μg of LPS was administered to each side of the hippocampus of 7-day-old mice to induce schizophrenia. Thereafter, 100 μg/kg of the nanobody (C3) of the present invention was intraperitoneally administered to 56-day-old adult mice once daily, and this process was repeated for one week. A week later, a schizophrenia-related behavioral experiment was conducted.

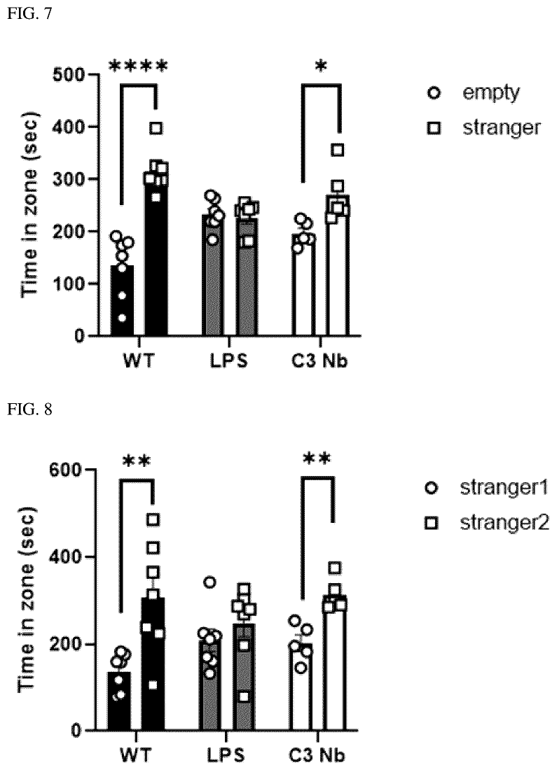

As a result, it was confirmed that when a 3-chamber test was performed for negative symptoms, the LPS-administered rats tended not to distinguish between an empty cup and a stranger mouse cup, but the groups to which the nanobody of the present invention was administered showed a significant difference in recovery (FIG. 7).

### Example 7: Confirmation of Effect of Nanobody of the Present Invention on Negative Symptoms in Animal Model of Schizophrenia (in vivo), Social Recognition (2)

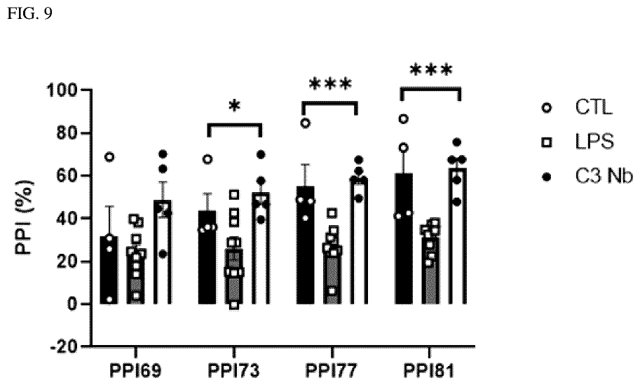

Following the behavioral experiment for negative symptoms in Example 6, an additional behavioral experiment was performed. The additional behavioral experiment was an experiment to check social recognition ability and to distinguish between a stranger mouse and a new stranger in Example 6. In this case, it was confirmed that the LPS animal model did not see the difference between the two, whereas the group to which the nanobody (C3) of the present invention was administered intraperitoneally for a week showed significant recovery (FIG. 8).

### Example 8: Confirmation of Effect of Nanobody of the Present Invention on Positive Symptoms in Animal Model of Schizophrenia (in vivo)

Following the behavioral experiment for negative symptoms in Example 6, an additional behavioral experiment was performed. This was an experiment to examine positive symptoms in the same animal model of schizophrenia as in Example 6 and check prepulse inhibition (PPI). In this case, it was confirmed that the LPS animal model showed prepulse inhibition impairment, whereas the groups to which the nanobody (C3) of the present invention was administered intraperitoneally for a week showed significant recovery (FIG. 9).

The nanobody or antigen binding fragment thereof according to the present invention binds to proteins (i.e., C1, C3, or C4) of the complement system to inhibit or interfere with their activity, thereby preventing a decrease in dendritic spines in nerve cells, and thus effectively improving the positive and negative symptoms of schizophrenia. In particular, the nanobody or antigen binding fragment thereof according to the prevent invention has an excellent advantage in that it can improve the negative symptoms of schizophrenia, which were difficult to treat in the past.

Although specific parts of the present invention have been described in detail, it will be obvious to a person with ordinary skill in the art that such a specific description is merely a preferred embodiment and the scope of the present invention is not limited thereby. Accordingly, the actual scope of the present invention will be defined by the appended claims and equivalents thereof.

What is claimed is:

1. A nanobody or an antigen binding fragment thereof comprising:
(a) heavy-chain CDR1 including a sequence of SEQ ID NO: 1, heavy-chain CDR2 including a sequence of SEQ ID NO: 2, and heavy-chain CDR3 including a sequence of SEQ ID NO: 3; and light-chain CDR1 including a sequence of SEQ ID NO: 4, light-chain CDR2 including a sequence of SEQ ID NO: 5, and light-chain CDR3 including a sequence of SEQ ID NO: 6,
(b) heavy-chain CDR1 including a sequence of SEQ ID NO: 7, heavy-chain CDR2 including a sequence of SEQ ID NO: 8, and heavy-chain CDR3 including a sequence of SEQ ID NO: 9; and light-chain CDR1 including a sequence of SEQ ID NO: 10, light-chain CDR2 including a sequence of SEQ ID NO: 11, and light-chain CDR3 including a sequence of SEQ ID NO: 12,
(c) heavy-chain CDR1 including a sequence of SEQ ID NO: 13, heavy-chain CDR2 including a sequence of SEQ ID NO: 14, and heavy-chain CDR3 including a sequence of SEQ ID NO: 15; and light-chain CDR1 including a sequence of SEQ ID NO: 16, light-chain CDR2 including a sequence of SEQ ID NO: 17, and light-chain CDR3 including a sequence of SEQ ID NO: 18, or
(d) heavy-chain CDR1 including a sequence of SEQ ID NO: 19, heavy-chain CDR2 including a sequence of SEQ ID NO: 20, and heavy-chain CDR3 including a sequence of SEQ ID NO: 21; and light-chain CDR1 including a sequence of SEQ ID NO: 22, light-chain CDR2 including a sequence of SEQ ID NO: 23, and light-chain CDR3 including a sequence of SEQ ID NO: 24.

2. The nanobody or antigen binding fragment thereof of claim 1, wherein the nanobody binds to any one or more selected from the group consisting of complement 1 (C1), complement 3 (C3), and complement 4 (C4).

3. The nanobody or antigen binding fragment thereof of claim 1, wherein the nanobody passes through a blood-brain barrier.

4. The nanobody or antigen binding fragment thereof of claim 1, wherein the nanobody prevents a decrease in dendritic spines in nerve cells.

5. The nanobody or antigen binding fragment thereof of claim 1, wherein the Fc region is removed.

6. The nanobody or antigen binding fragment thereof of claim 1, wherein the nanobody or antigen binding fragment inhibit activity of C1, C3 or C4.

7. A nanobody-drug conjugate in which a drug is bound to the nanobody or antigen binding fragment thereof of claims 1.

8. A pharmaceutical composition for preventing or treating a mental or neurological disease, comprising the nanobody or antigen binding fragment thereof of claims 1.

9. The pharmaceutical composition of claim 8, wherein the mental disease comprises any one or more selected from the group consisting of schizophrenia, depression, anxiety disorders, post-traumatic stress disorder (PTSD), panic disorders, autism spectrum disorders, and attention deficit/hyperactivity disorder (ADHD).

10. The pharmaceutical composition of claim 8, wherein the neurological disease comprises any one or more selected from the group consisting of Alzheimer's disease, mild cognitive impairment, and dementia.
